(19) United States
(12) Patent Application Publication
(10) Pub. No.: US 2004/0023841 A1
(43) Pub. Date: Feb. 5, 2004

(54) Combinatorial method for rapid screening of drug delivery formulations

(76) Inventors: Samir Mitragotri, Goleta, CA (US); Pankaj Karande, Santa Barbara, CA (US)

Correspondence Address: FULBRIGHT AND JAWORSKI L L P, PATENT DOCKETING 29TH FLOOR, 865 SOUTH FIGUEROA STREET, LOS ANGELES, CA, 900172576

(21) Appl. No.: 10/344,961

(22) PCT Filed: Aug. 23, 2001
(86) PCT No.: PCT/US01/26473

(51) Int. Cl. A61K 31/00; G01N 33/53; G01N 33/543

(52) U.S. Cl. 514/1; 435/7.1; 436/518

(57) ABSTRACT

This invention describes a novel method for rapid screening of formulations for drug delivery, which uses a high throughput array to screen multiple samples. This method monitors the depletion of a test substance from a donor well, the migration of the substance into a test membrane, and/or the migration of the substance through a membrane into a receptor well. It can be used to discover new formulations as well as to optimize the existing formulations for delivering drugs via transdermal, oral, or injectable routes.

### I. CROSS REFERENCE TO RELATED APPLICATIONS

This application claims the benefit of Provisional Patent Application No. 60/227,453, filed Aug. 23, 2000, which is incorporated herein by reference.

### II. BACKGROUND OF THE INVENTION

A. Field of the Invention

The field of the invention is screening methods for drug delivery.

B. Description of Related Art

Recent advances in biotechnology have allowed rapid screening of thousands of drugs for their effectiveness; see Ng, et al., infra; Verdine, et al., infra. Through the development of combinatorial drug discovery, new drugs, especially low-molecular weight analogs of proteins and peptides, are being continually developed; see Zhang, et al., infra. However, the ability to deliver these drugs is still evaluated by the traditional experiments. In these experiments, the biological membrane under consideration, such as the skin for transdermal drug delivery or the intestine for oral drug delivery, is placed in a diffusion cell and the transport across this membrane is measured over several hours; see Bronaugh, et al., infra. In many cases, additional experiments are performed to assess the effect of formulation on membrane permeability. During this process, various formulations are utilized to optimize drug bioavailibility. The objective of this optimization is to identify a formulation that can deliver the required therapeutic dose into the body. Only a few drugs pass this test and are then transferred to the next stage of development. This process is based on traditional experiments and is time-consuming as well as expensive. Availability of a rapid screening method to determine trans-membrane transport of drugs should facilitate the development of drug delivery systems. In spite of their potential value, such methods are not currently available.

A typical drug formulation may contain anywhere from 3-15 components. For example, consider a formulation containing six components, including the drug. In order to optimize the concentration of these components, an experimental design is required, for example, five levels of concentration of each component. In order to determine the optimal concentration of these components, 5⁶ experiments are required; that is, about 15,000. Note that in a typical formulation development project testing of a system containing more than six components is not unusual. Thus, the number of experiments required for optimization is extremely large. This problem is circumvented by reducing the parameter space by either eliminating some of the components or by reducing the levels of each component in the experimental design. Although this process reduces the number of experiments needed to be done, it greatly increases the likelihood of “missing” important formulations. This is especially important since the interactions between the components of the formulation are very complex and are difficult to predict. A typical transdermal transport experiment lasts for at least 24 hours and uses about a 2 cm piece of skin. It is customary to run about 15-20 transport experiments at a time. At this rate, it would take hundreds of days to screen all 15,000 combinations. Thus, it is extremely difficult to perform these many experiments. Hence, intuition is used to eliminate a majority of these combinations. Although this decreases the number of experiments, it increases the risk of not finding a valuable formulation.

Applications of transdermal drug delivery are limited by low skin permeability. More than 250 chemicals have been identified as potent enhancers in the literature that can increase skin permeability. In spite of this only a few of these enhancers are actually used in practice due to low efficacy and irritation. Also different enhancers interact with the skin via different mechanisms to increase skin permeability. In absence of the fundamental knowledge of these interactions, we need to rely on a rapid method to screen various enhancers.

### III. SUMMARY OF INVENTION

The disclosed invention offers a method that greatly increases the efficiency of formulation screening. We hypothesize that combinations of enhancers work better as compared to individual enhancers alone. It becomes increasingly difficult to screen efficiency of formulations with increasing number (two or more) of enhancer components. To test the efficiency of a multitude of such formulations that result from having all possible enhancer combinations, in an efficient and expeditious way, we take recourse to High Throughput screening.

The method for performing high throughput assays of drug delivery formulations includes the steps of:

i) securing a test membrane to a device comprising a donor plate, which includes a plurality of donor wells formed by donor holes passing through the donor plate;

ii) introducing a formulation into each donor well, the formulation including a test substance and an inert medium; and

iii) evaluating a characteristic of the test substance that remains in the donor well or migrates into the test membrane.

The test membrane is preferably mammalian skin or mucosa. The device may also include a receiver plate having a plurality of receptor wells corresponding to the donor wells of the donor plate, so that the test membrane is secured between the donor plate and the receiver plate. In addition, the device may further comprise two electrodes to measure current across the membrane. Preferably, the donor holes have a diameter of about 40 microns to about 10 mm and the volume of the donor wells is about 1 to 500 R.

Formulations for testing include a test substance within an inert medium. Typically, the test substance is a drug or its analog. A preferred drug analog is a molecule of similar size and chemical properties to the drug, which is also a radioactive or fluorescent substance. Preferred formulations also include one or more permeability enhancers The inert medium can be an ointment, cream, gel, solution or lotion.

The concentration of test substance that remains in the donor well can be determined by conventional assays, such as HPLC, UV spectroscopy and the like. A test substance that migrates into the test membrane can be monitored by its radioactivity, fluorescence or enhancement of membrane conductivity. Such evaluating steps can recur at one or more periodic intervals over a period of about 24 hrs. Preferably the evaluating step occurs no later than 8 hours after introducing the formulation to the donor well.

The present invention also includes a device for conducting high throughput assays of drug formulations, which includes a donor plate having a plurality of donor wells formed by donor holes passing through the donor plate; means for securing a test membrane to the donor plate, whereby one or more donor wells are sealed at one end of the well and transfer of one or more substances to the membrane can occur; and one or more electrodes to measure current across a portion of the test membrane, said portion sealing an individual donor well.

These and other features, aspects, and advantages of the present invention will become better understood with regard to the following detailed description and accompanying drawings.

### IV. BRIEF DESCRIPTION OF THE DRAWINGS

FIG. 1 is a schematic representation of the device used for formulation testing, FIG. 1IA is a side view of the disc used for screening transdermal formulations, FIG. 1B is a top-view representation of the disc of FIG. 1A;

FIG. 2 is a schematic representation of the skin and the stratum corneum (SC) layer;

FIG. 3 is a graph showing the loss of drug from the donor hole into the skin;

FIG. 4 is a graph showing the relationship between the percent drug lost from the donor hole versus permeabilities measured by traditional methods that use diffusion cells;

FIG. 5 is an image of the skin obtained during the experiment performed using the device shown in FIG. 1;

FIG. 6 is a graph representing sulforhodamine delivery with varying ratios of SLS and dodecyl pyridinium chloride, corresponding to the image shown in FIG. 5;

FIG. 7A shows a top view of a 10×10 High Throughput Array assembly, where the top and bottom plate are held together by a set of four screws;

FIG. 7B. shows and oblique view of the 10×10 High Throughput Array assembly.

FIG. 8 shows Conductivity Enhancement for SLS using 10×10 High Throughput array;

FIG. 9 shows Conductivity Enhancement for TDAB using 10×10 High Throughput array;

FIG. 10 shows Transport Enhancement for SLS and TDAB using 10×10 High Throughput array at the end of 7 Hrs;

FIG. 11 shows Conductivity Enhancement for SLS using 5×5 High Throughput array;

FIG. 12 shows Conductivity Enhancement for TDAB using 5×5 High Throughput array;

FIG. 13 shows Transport Enhancement for SLS and TDAB using 5×5 High Throughput array at the end of 7 Hrs;

FIG. 14 shows Conductivity Enhancement for SLS using Franz Diffusion cells;

FIG. 15 shows Conductivity Enhancement for TDAB using Franz Diffusion cells;

FIG. 16 shows Transport Enhancement for SLS and TDAB using Franz Diffusion cells at the end of 7 Hrs;

FIG. 17 shows the Effectiveness ratio (SLS: TDAB) for Franz diffusion cell and 10×10 HTP array at 18 Hrs and 25 Hrs;

FIG. 18 shows Average conductivity enhancement for SLS over three different geometries;

FIG. 19 shows Average conductivity enhancement for TDAB over three different geometries;

FIG. 20 shows Average transport enhancement for SLS and TDAB over three different geometries; and

FIG. 21 shows Conductivity (current per unit area) in Franz Diffusion cells and High Throughput array (10×10).

### V. DETAILED DESCRIPTION

Conventional methods to study transdermal transport using Franz diffusion cells are limited in their efficiency as they can typically perform one test per square inch of the skin. The hold up time in these cells is about 48 hrs. We propose a novel High Through Put (HTP) screening method that can perform about 5 to 300 tests per square inch of skin. The hold up times with HTP screening method are also lower than in the Franz diffusion cells (about 7 hrs as against 48 hrs). This results in a method that can be at least 50-400 times more efficient than conventional techniques. This is a great improvement in the efficiency of formulation testing.

A. HTP Screening Array

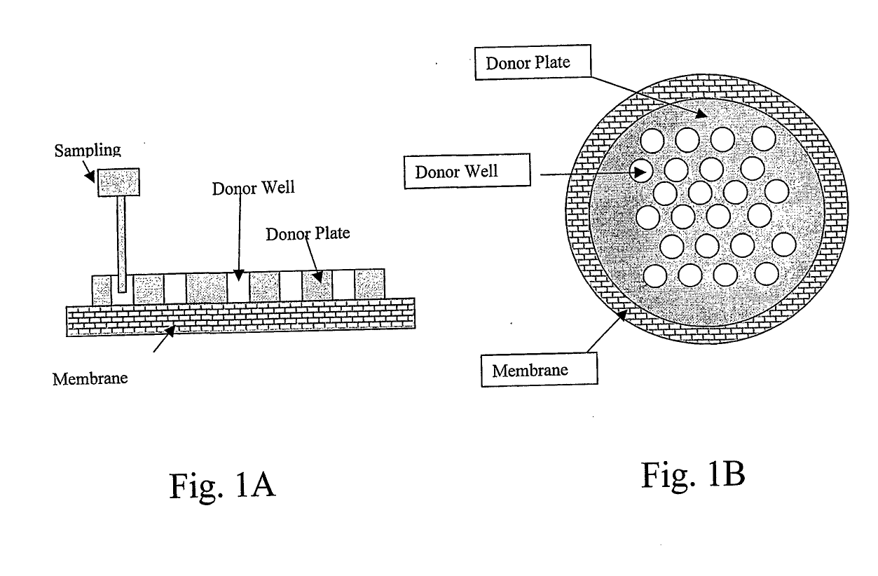

The proposed invention makes use of a HTP screening array for determining the effectiveness of formulations for drug delivery. A schematic representation of a device for screening transdermal formulations is shown in FIG. 1A and FIG. 1B. FIG. 1A shows a cross-sectional view of the device that will be used in the disclosed method. The device, as shown in FIG. 1A, consists of a disc, which can be made from teflon, polycarbonate, silicon or other material; about 5 cm in diameter and about 0.5-2 mm in height. The disc contains about 600 holes, each hole having a diameter of about 2 mm, as shown in a top-view in FIG. 1B. The disc is placed on the skin. The holes are subsequently filled with formulations to be tested.

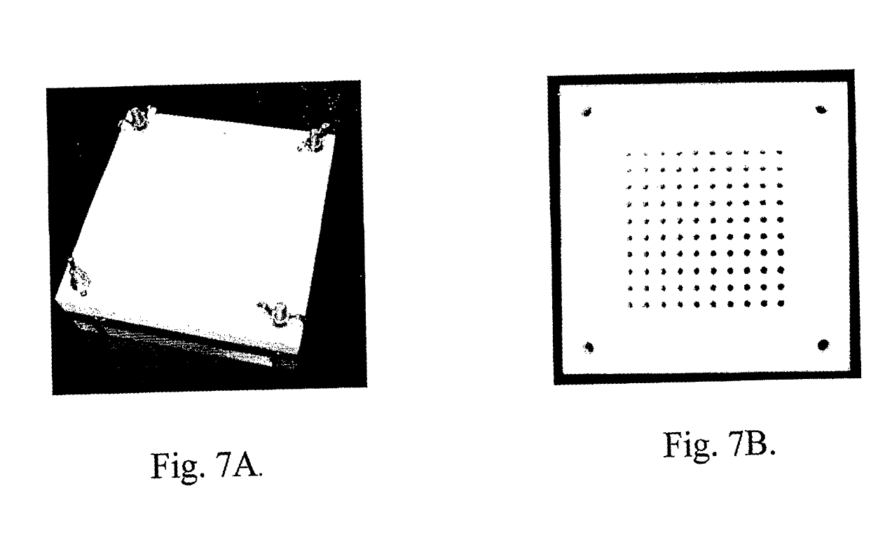

Another HTP screening array, which mimics the Franz diffusion cells on a miniature scale, consists of two polycarbonate plates each 0.5 inches thick. The top plate (donor plate) has through holes (wells) drilled in it, each of which acts as an isolated donor chamber similar to the donor chamber in the Franz diffusion cells. The bottom plate (receiver plate) also has holes (wells) drilled in the same pattern as the donor plate and simulates the receiver compartment of the diffusion cells. All wells are isolated from each other for all practical purposes and each well acts like an individual diffusion cell. The skin is placed between the donor and receiver plate and the plate assembly is clamped using four screws as shown in the figure (FIG. 7A)

The wells in the receiver plate are filled with PBS. The skin is placed on the receiver plate with the stratum corneum (SC) facing the donor plate. The donor plate is then placed on the skin and the entire assembly is clamped tightly using four screws. A mild vacuum is then applied to remove any excess PBS that may be pushed in to the wells in the receiver plate.

B. Membrane

Although the present invention is primarily directed at transdermal delivery applications, this method can extend to other drug delivery methods including oral delivery. For example, this method can be used to screen formulations for oral drug delivery by replacing the skin with mucosal membrane.

The test membrane can be any of a variety of membranes suitable for use in the diffusion experiments, such as hairless mouse skin, porcine skin, guinea pig skin, human skin, or alternatively, a synthetic membrane may be used, such as an elastomeric membrane, or any of a number of endothelial or epithelial cell culture barriers, such as those described in Audus, K. L., et al., Pharmaceutical Research, 1990, 7 (5), p 435. Screening of formulations for transdermal delivery is most preferably conducted using pigskin.

A typical transdermal transport experiment lasts for at least 24 hours and uses about a 2 cm² piece of skin. In contrast, the method of the present invention uses as little as 0.03 cm² of skin per experiment, which is a much smaller area compared to the traditional methods that use about 1-2 cm² of skin. Moreover, because a smaller surface area is utilized the cost of experiments is also reduced as well as the amount of chemicals used for screening.

1. Formulations

A typical drug formulation may contain anywhere from 3-15 components. In order to prepare the formulations, its components are first identified. For example, a formulation generally includes a test substance, typically a drug or a drug analog, within a an inert medium. The drug analog can be a molecule of about the same size and chemical properties of the drug, which may include a radioactive tracer or fluorescent dye for ease of detection. The inert media can include any of a number of solvents, carriers, binders, gelling agents, and so forth, for an active agent to be delivered. Media for topical delivery include ointments, creams, gels, solutions and lotions. While ointments are composed of mostly high molecular weight hydrocarbons, creams, gels, solutions and lotions typically comprise up to 90 percent of fairly volatile solvents, such as water, ethanol and propylene glycol.

Preferably, the formulation will include one or more permeability enhancers. Over 250 enhancers have been used for enhancing transdermal drug transport. These enhancers have been classified into several categories based on their structure or their effect on permeability:

Surfactants: These are amphiphilic molecules with a hydrophilic head and a hydrophobic tail group. The tail length and the chemistry of the head group play an important role in determining their effect on skin permeability. Surfactants can be categorized into four groups, cationic, anionic, non-ionic, and zwitter-ionic depending on the charge on the head group. Prominent examples of surfactants that have been used for transdermal delivery include: Brij (various chain lengths), HCO-60 surfactant, Hydroxypolyethoxydodecane, Lauroyl sarcosine, Nonionic surface active agents, Nonoxynol, Octoxynol, Phenylsulfonate, Pluronic, Polyoleates (nonionic surfactants) Rewopal HVIO, Sodium laurate, Sodium oleate, Sorbitan dilaurate, Sorbitan dioleate, Sorbitan monolaurate, Sorbitan monooleates, Sorbitan trilaurate, Sorbitan trioleate, Span 20, Span 40, Span 85, Synperonic NP, Triton X-100, Tweens, Sodium alkyl sulfates, and alkyl ammonium halides.

Azone and related compounds: These compounds are also amphiphilic and possess a nitrogen molecule in their head group (preferable in the ring). The presence of a nitrogen atom makes these surfactants very peculiar in terms of their interactions with skin. Examples of such compounds include N-Acyl-hexahydro-2-oxo-1H-azepines, N-Alkyl-dihydro-1,4-oxazepine-5,7-diones, N-Alkylmorpholine-2,3-diones, -Alkylmorpholine-3,5-diones, Azacycloalkane derivatives (-ketone, -thione).

Solvents and related compounds: These molecules are solubility enhancers. Some of them also extract lipids, thereby increasing skin permeability. Examples of solvents include Acetamide and derivatives , Acetone, n-Alkanes (chain length between 7 and 16), Alkanols, diols, short-chain fatty acids , Cyclohexyl-1,1-dimethylethanol, Dimethyl acetamide, Dimethyl formamide, Ethanol, Ethanol/d-limonene combination, 2-Ethyl-1,3-hexanediol, Xylene, DMSO and related compounds.

Fatty alcohols, fatty acids, fatty esters, and related structures: Thse molecules are classic bilayer fluidizers. These correspond to one of the most investigated class of enhancers. Examples of these enhancers include Aliphatic alcohols, Decanol, Lauryl alcohol (dodecanol), Linolenyl alcohol, Nerolidol, 1-Nonanol, n-Octanol, Oleyl alcohol, Butyl acetate, Cetyl lactate, Decyl N,N-dimethylamino acetate, Decyl N,N-dimethylamino isopropionate, Diethyleneglycol oleate, Diethyl sebacate, Diethyl succinate, Diisopropyl sebacate, Tetradecyl N,N-dimethylamino, Sodium deoxycholate, Sodium taurocholate, Sodium tauroglycocholate.

Others: Aliphatic thiols, Alkyl N,N-dialkyl-substituted amino acetates, Anise oil, Anticholinergic agent pretreatment, Ascaridole, Biphasic group derivatives, Bisabolol, Cardamom oil, I-Carvone, Chenopodium (70% ascaridole), Chenopodium oil, 1,8 Cineole (eucalyptol), Cod liver oil (fatty acid extract), 4-Decyloxazolidin-2-one, Dicyclohexylmethylamine oxide, Diethyl hexadecylphosphonate, Diethyl hexadecylphosphoramidate, N,N-Dimethyl dodecylamine-N-oxide, 4,4-Dimethyl-2-undecyl2-oxazoline, N-Dodecanoyl-L-amino acid methyl esters, 1,3-Dioxacycloalkanes, (SEPAs), Dithiothreitol, Eucalyptol (cineole), Eucalyptus oil, Eugenol.

Once the concentration levels of each component are chosen, formulations corresponding to these combinations are then prepared by mixing the components in the desired concentrations. About one to about 500 microliters of each formulation is placed in each donor well.

C. Sampling and Detection

With prior art permeation study testing procedures, the diffusion test is typically run for a period of 24 hours or more; over the course of the study, samples are periodically withdrawn from a receiver receptacle to evaluate the flux of drug through the skin over time. In contrast, the present invention may be designed so that the permeation experiment is run to a pre-determined end point, i.e., less than about eight hours. Depending on the drug, the formulation will typically remain on the membrane for several hours, which is referred to as contact time.

Immediately, or at the end of the contact time, a sample may be taken from the donor wells by an automated process and transferred to a detection device. Moreover, Donor samples may be periodically withdrawn from their respective wells, typically by aspiration, and assayed by an appropriate analytical method. For example, the samples can be assayed by any of a number of analytical test methods, such as HPLC (high performance liquid chromatography), UV (ultraviolet spectrometry), GC (gas chromatography), LC (liquid chromatography) or, if the samples are radiolabeled, scintillation counting.

Alternatively, disruption of the lipid bilayer, which also disrupts transport of ions, may be monitored by measuring the conductivity of skin. Accordingly, one embodiment of the present invention uses electrodes to measure the conductivity of the skin, which is proportional to permeability. The conductivity measurements may be conveniently taken at periodic intervals without having to remove the formulation or disassemble the screening device.

In another embodiment, the receiver assembly is detached from the donor assembly upon termination of the diffusion experiment, and the membrane is assayed by fluorescence or liquid scintillation counting.

D. Analysis

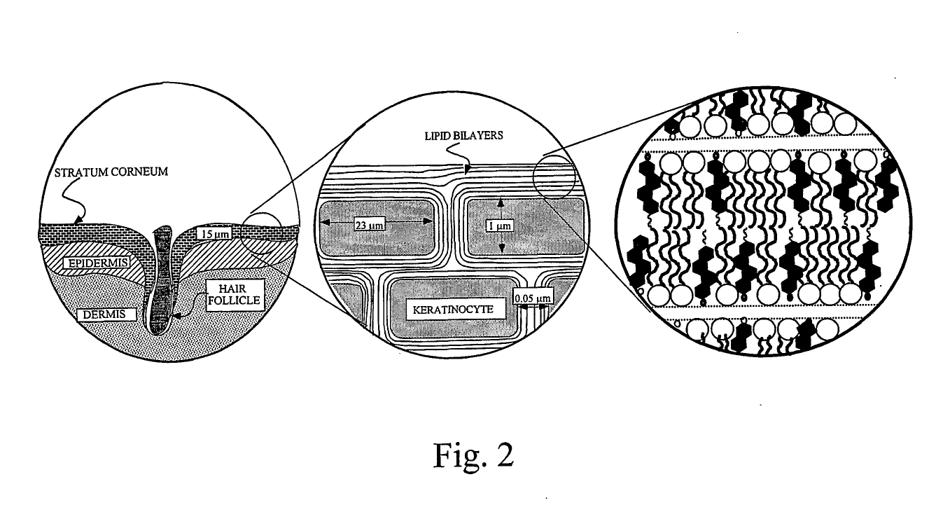

Stratum corneum, the uppermost layer of the skin, is the rate limiting step in transdermal transport. Stratum corneum consists of about 15 layers of keratin-filled cells called keratinocytes. In between the keratinocytes are lipid bilayers, as shown in FIG. 2. Low permeability of the SC is due to the low permeability of its lipid bilayers; see Mitragotri, et al., infra. Permeability of SC to drugs is determined by two important transport coefficients: partition and diffusion coefficients; see Johnson, infra. Solute diffusion into the stratum corneum (SC) is described by Fick's law, Eq. 1 as follows; see Crank, infra:

$$\frac{\partial C}{\partial t} = - D \frac{{\partial }^{2} C}{\partial {x}^{2}} \qquad [ 1 ]$$

where, D is the solute diffusion coefficient, C is the solute concentration, and x is the distance. As discussed earlier, the SC consists of layers of kceratinocytes and intracellular spaces filled with lipid bilayers; see Elias, et al., infra. Transdermal transport of drugs, especially hydrophobic drugs, occurs through these lipid bilayers. Hence, only a small fraction of the area is available for drug transport. Furthermore, the drug has to follow a tortuous path to cross the SC. So the effective SC thickness for solute transport is τ*L, where, L is the SC thickness and τ* is the effective tortuosity factor.

During the contact time, the solute concentration in the formulation decreases over time due to drug penetration into the SC. Specifically, the concentration of the solute in the formulation, C(t), decreases with time before reaching an equilibrium value, C_{∞}. The rate of drug release is determined by Fick's law as described in Eq. [1]. This equation can be solved to arrive at the following equation; see Crank, infra:

$$\begin{matrix}\frac{[ C ( 0 ) - C ( t ) ]}{{C}_{∞}} = \frac{1}{α} ( 1 - {\sum }_{n = 0}^{∞} \frac{2 α ( 1 + α )}{1 + α + {α}^{2} {q}_{n}} {ⅇ}^{- {D}_{h} {q}_{n}^{2} t / {( L {τ}^{*} )}^{2}} ) & [ 2 ] \\ where , α = \frac{{V}_{PBS}}{{K}_{b} {V}_{SC} f} & [ 3 ]\end{matrix}$$

where, V_{PBS} is the thickness of the formulation layer on the SC. V_{SC} is the volume of the SC used for experiments (V_{SC}=L×Area) (typically 4.7×10⁻⁵ cm³, that is, Area=0.0314 cm² and L=15 μm; see Mitragotri, et al., infra), and f is the fractional volume of lipids in the SC (0.1; see Mitragotri, et al., infra). Equation [2] can be simplified for short times as follows; see Crank, infra:

$$\frac{[ C ( 0 ) - C ( t ) ]}{{C}_{∞}} = ( 1 + \frac{1}{α} ) ( 1 - {ⅇ}^{\frac{{D}_{b} t}{{( L {τ}^{*} )}^{2} {α}^{2}}} {erfc [ \frac{{D}_{b} t}{{( L {τ}^{*} )}^{2} {α}^{2}} ]}^{0.5} ) \qquad [ 4 ]$$

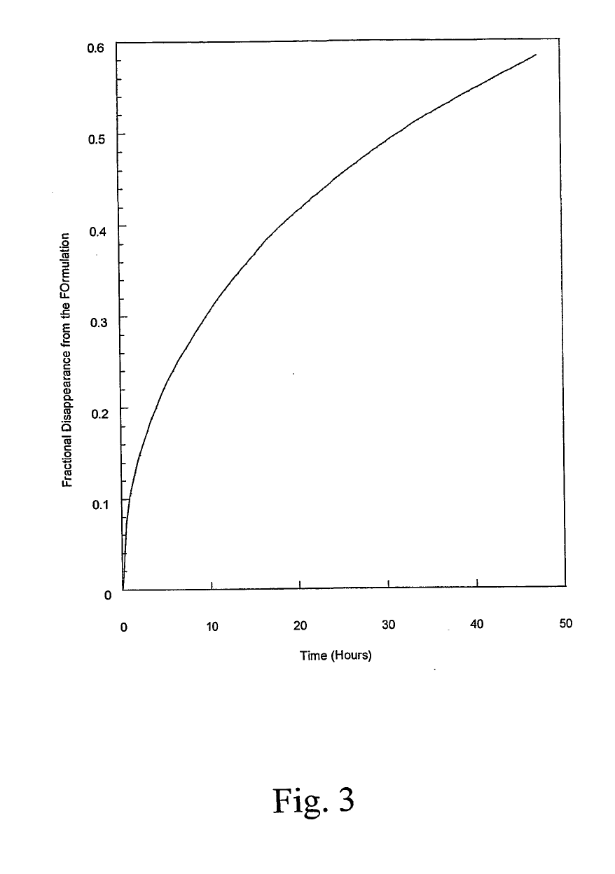

For example, FIG. 3 is a typical plot of the left hand side of Eq. [4]. FIG. 3 shows that the amount of drug lost from the donor hole increases with time before achieving equilibrium at times greater than 50 hours.

The equations described above [Eq.1-4] are used to determine the effectiveness of formulations via three methods. In the first method, the effectiveness of the formulation is determined by the loss of the drug from the formulation in a given amount of time. Specifically, the higher the loss of the drug from the formulation in a given amount of time, the higher the penetration of the drug into the skin. In the second method, the amount of drug delivered into the skin is measured by radioactivity, fluorescence or conductivity assays. In the third method, which corresponds to the traditional method, the amount of drug delivered across the skin is measured and used to determine the most effective formulation.

E. Advantages of using HTP Screening.

As described above, the HTP method can be at least 50-400 fold more efficient as compared to conventional Franz diffusion cells on basis of skin area utilized, sampling volume and hold up times. Moreover, there is no physical, experimental or fundamental limit on the size of wells used in the HTP array. We can scale down to a smaller well diameter and correspondingly further increase the efficiency of the HTP screening method. Once we have established the efficacy of an enhancer in increasing conductivity of the skin, we can find out the actual amount of drug transported across the skin using Franz diffusion cells. Knowing the relative efficiency of one enhancer over another from HTP screening, it is sufficient to repeat transport experiments in Franz diffusion cells for only one enhancer at one concentration. Thus we conclude that the novel HTP screening method we propose is a much more efficient way of screening enhancers. It is not only useful as a tool for identifying the right vehicle for transdermal drug delivery but also a means to answer some fundamental underlying issues of transdermal transport.

### EXAMPLES

### Example 1

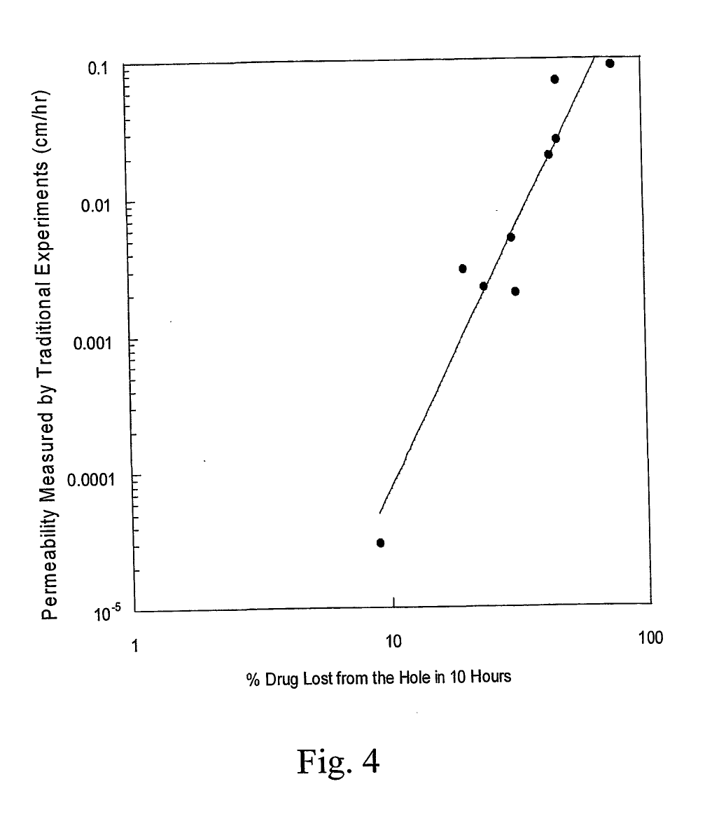

The following experiments were performed to assess the usefulness of a method, which determines the loss of a test substance from a formulation over time. Experiments were performed to assess whether this method can be used for estimation of skin permeability to drugs. For these studies, phosphate buffered saline was used as a model formulation. A disc, as shown in FIG. 1, was prepared from Teflon. Ten holes were drilled into the disc and each hole was about 2 mm wide. The disc was then placed on the SC prepared from human skin. Solutions of nine different drugs in PBS were placed in various holes. The concentration of drugs in the solutions were measured at time zero. The drugs were allowed to remain in contact with the skin for 24 hours. Samples were taken from the holes at the end of 24 hours and then analyzed using a liquid scintillation counter. The amount of drug lost from the donor hole was calculated. Permeability measurements of the same drugs were performed using conventional methods that use macroscopic diffusion cells. The results of the experiment are shown in Table I. Moreover, FIG. 4 shows that the drug lost from the hole was proportional to the permeability measured by traditional methods that use diffusion cells. Hence, the amount of drug lost can be predictive of drug permeability. This is very important since the amount of drug lost can be measured quickly using a device shown in FIG. 1.

TABLE I

| | [symbol] % Drug Lost from the | Permeability Measured by |
|---|---|---|
| Drug | Hole | Traditional Experiments |
| Butanol | 32% | 2.0 E-03 |
| Hexanol | 31% | 5.0 E-03 |
| Octanol | 47% | 7.0 E-02 |
| Octadecanol | 77% | 9.0 E-02 |
| Testosterone | 24% | 2.2 E-03 |
| Aldosterone | 9% | 3.0 E-05 |
| Progesterone | 44% | 2.0 E-02 |
| Napthalene | 47% | 2.6 E-02 |
| Lidocaine | 20% | 3.0 E-03 |

### Example 2

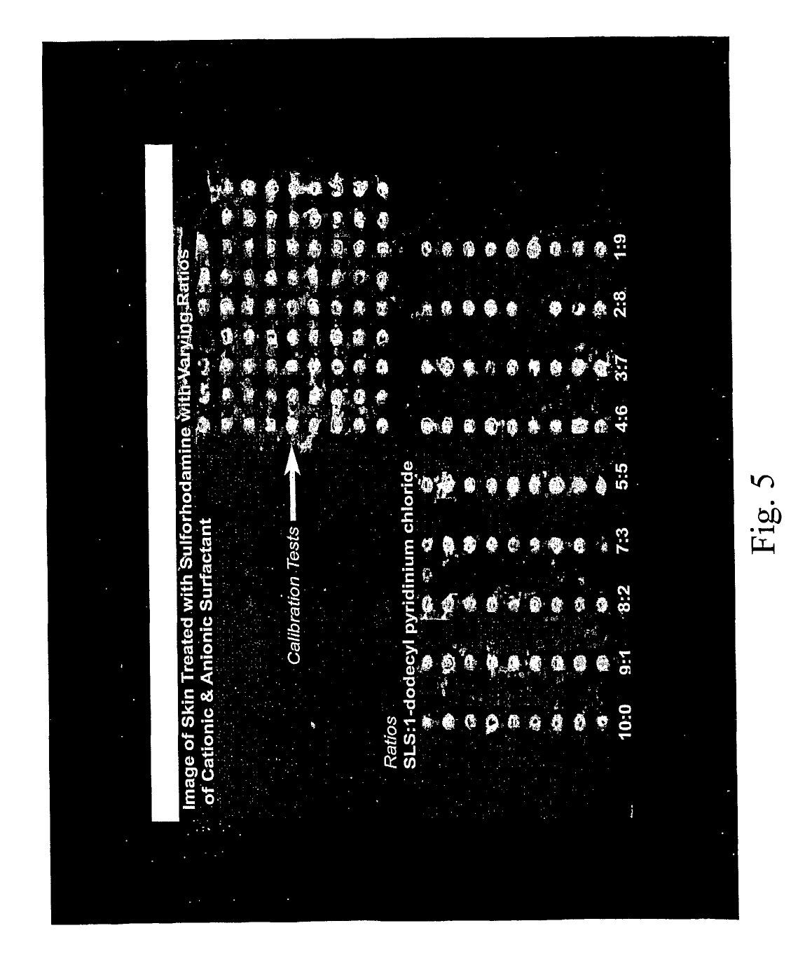

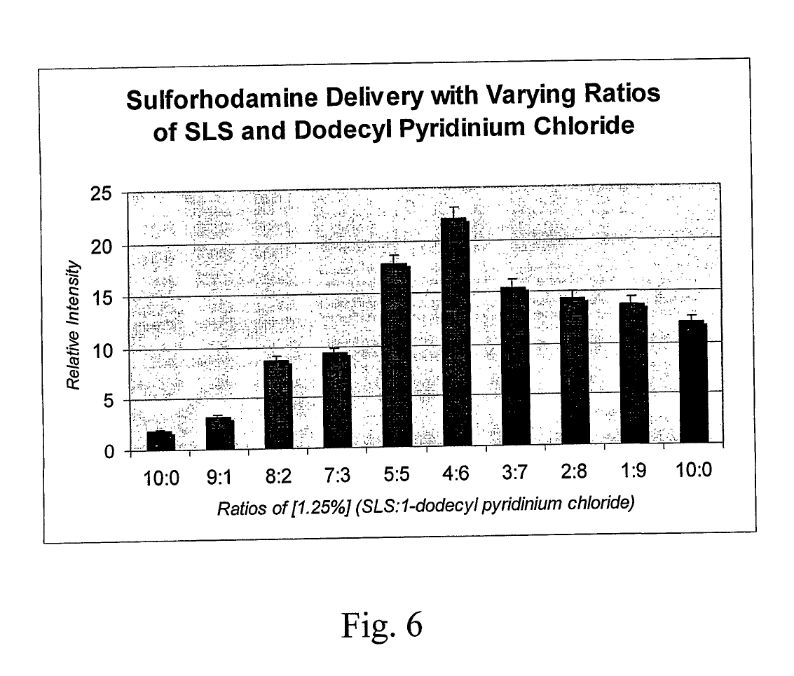

The following example describes a method where the amount of drug measured in the skin can be used to screen formulations. For this purpose, a device shown in FIG. 1 was prepared using plexi glass. The device was configured in a square shape. The device consisted of two plates each having 400 holes each with a diameter of about 700 micron. A sample of pigskin was sandwiched between the two plates and the plates were clamped. The holes were filled with formulations to be tested for drug delivery. Two model drugs (fluorescein and sulforhodamine) were used to assess the efficacy of the enhancers. Two model enhancers, sodium lauryl sulfate and dodecyl pyridinium chloride, were used in these experiments. Various combinations of these two enhancers were prepared by mixing these enhancers. The objective of these tests was to find out whether the combination of these two enhancers is more effective than each of them alone in enhancing transdermal transport. We prepared various combinations of these enhancers and filled them in holes along with model drugs listed above. The formulations were allowed to remain in contact with skin for 20 hours. At the end of the incubation time, the skin was removed from the device and observed under bright light. Images were taken using a digital camera. An example of the image is shown in FIG. 5. The areas that appear bright correspond to greater delivery of drug into the skin. The image shown in FIG. 5 was analyzed and quantified to assess which enhancers are more effective. This data is shown in FIG. 6, which shows the variation of the amount of drug delivered as a function of formulation composition. FIG. 6 shows that the formulation, a mixture of sodium lauryl sulfate and dodecyl pyridinium chloride (4:6 parts), is most effective in delivering drugs. Thus, the disclosed method allows discovery of new formulations for effective drug delivery.

### Example 3

To validate the high throughput screening method we made use of two additional screening arrays. Two model enhancers were selected, Sodium Lauryl Sulfate (SLS) and Tetra Decyl Ammonium Bromide (TDAB). Several different formulations of these enhancers were prepared in PBS at varying total surfactant concentrations from 0% (w/v) to 2% (w/v). Similar experiments were performed using both the arrays. A description of the experimental details and results follows.

2. 10×10 Array.

This array is built as a pattern of 10×10 matrix. This corresponds to 100 test wells, each well 3 mm in diameter.

a. 1.A. Conductivity Measurements.

The formulations to be tested are filled in the donor compartments. Each formulation is filled in 4 wells and each well can hold about 85 μL of the test formulation. Two 22 G 1½ needles are used as electrodes to measure current across the skin. One needle is stuck into the dermis and acts as the common electrode while the other needle is sequentially placed in each well to measure current. Current measurements are made across the skin periodically over a span of 25 hrs. The current, measured at 100 Hz and 143 mVpp, varied between 1 HA at time 0 to 10-12 μA at time 25 hrs.

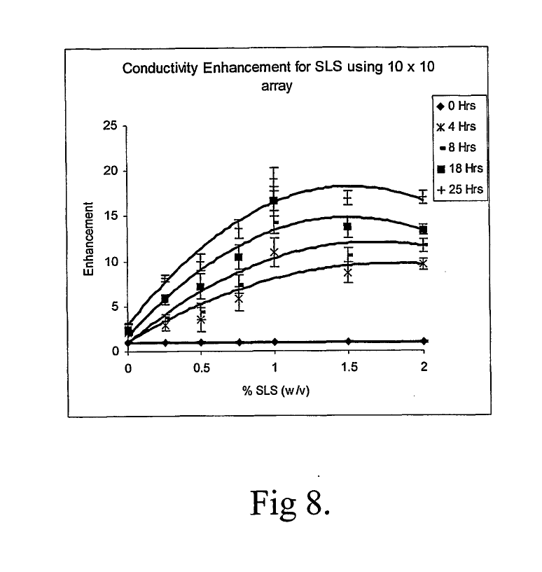

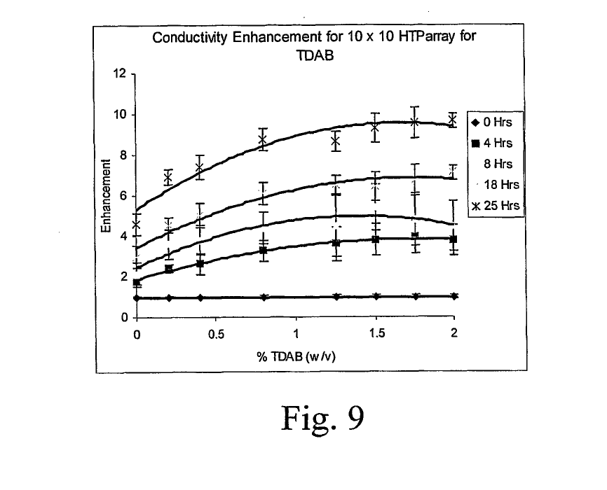

The conductivity enhancement for a given formulation at time ‘t’ is then calculated as

$${E}_{t}^{C} = \frac{{I}_{t}}{{I}_{0}} ,$$

where Iₜ is the current across the skin at time ‘t’ and I₀ is the current across the skin at time 0. Conductivity enhancement for SLS (FIG. 8) and TDAB (FIG. 9) is plotted at various times for different concentrations between 0% (w/v) to 2% (w/v). The conductivity enhancement increases with increasing SLS or TDAB concentration and reaches a maximum after which it starts decreasing. This effect gets more pronounced at larger times. The location of the maximum on the enhancement curve is a function of time. The error bars correspond to the standard deviations. (n=4).

b. Radiation Measurements.

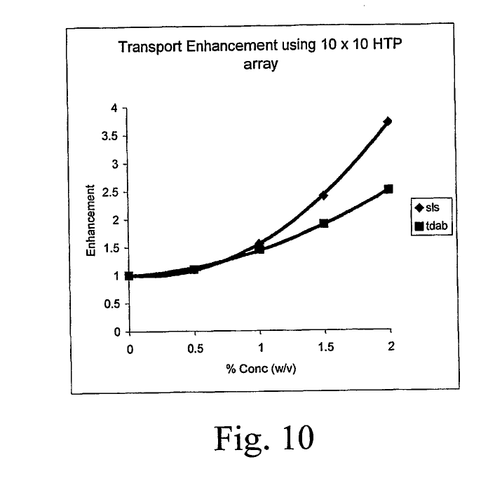

Radiolabelled mannitol was added to all formulations prepared in PBS at a concentration of 10 μCi/mL. The donor compartments were filled with these formulations with each formulation filled in 4 wells. The skin was then incubated for 7 hrs. The solutions from the donor compartment were removed at the end of incubation period. The skin was then gently rinsed to free any mannitol that could be sticking to the surface of the skin. The skin was then cut and dissolved in 0.5 M Solvable, a tissue and gel solubilizer from Packard Chemicals, at 60° C. overnight. A 500 μL sample was then taken and concentration of radiolabeled mannitol in this sample was then measured using a scintillation counter (Packard Tricarb 2000 CA). The transport enhancement at different test formulations is then calculated as

$${E}^{T} = \frac{{C}_{F}}{{C}_{C}} ,$$

where C_{F} is the radiation count for a particular test formulation and C_{C} is the radiation count for the control i.e. PBS alone without any enhancer. Transport enhancement for SLS and TDAB (FIG. 10) is plotted at the end of 7 Hrs for different concentrations between 0% (w/v) to 2% (w/v). The amount of mannitol transported increases monotonously as a function of the surfactant concentration.

3. 5×5 Array.

This array is built as a pattern of a 5×5 matrix. This corresponds to 25 test wells, each well 7.5 mm in diameter. It consists of two polycarbonate plates each 0.5 inches thick. The top plate (donor plate) has 25 through holes (wells), diameter 7.5 mm, drilled in it, each of which acts as an isolated donor chamber similar to the donor chamber in the Franz diffusion cells. The bottom plate (receiver plate) also has holes (wells) drilled in a similar pattern as the donor plate and simulates the receiver compartment of the diffusion cells. All wells are isolated from each other for all practical purposes and each well acts like an individual diffusion cell. The skin is placed between the donor and receiver plate and the plate assembly is clamped using four screws as shown in the figure.

Screening of the formulations is performed using pigskin. The wells in the receiver plate are filled with PBS. The skin is placed on the receiver plate with the stratum corneum (SC) facing the donor plate. The donor plate is then placed on the skin and the entire assembly is clamped tightly using four screws. A mild vacuum is then applied to remove any excess PBS that may be pushed in to the wells in the receiver plate.

Tests were performed using the same model enhancers, Sodium Lauryl Sulfate (SLS) and Ammonium Bromide (TDAB). Several different formulations of these enhancers were prepared in PBS at varying total surfactant concentrations from 0% (w/v) to 2% (w/v).

a. Conductivity Measurements.

The formulations to be tested are filled in the donor compartments. Each formulation is filled in 2 wells and each well can hold about 500 μL of the test formulation. Two 22 G 1½ needles are used as electrodes to measure current across the skin. One needle is stuck into the dermis and acts as the common electrode while the other needle is sequentially placed in each well to measure current. Current measurements are made across the skin periodically over a span of 25 hrs. The current, measured at 100 Hz and 143 mVpp, varied between 1 μA at time 0 to 25-30 μA at time 25 hrs.

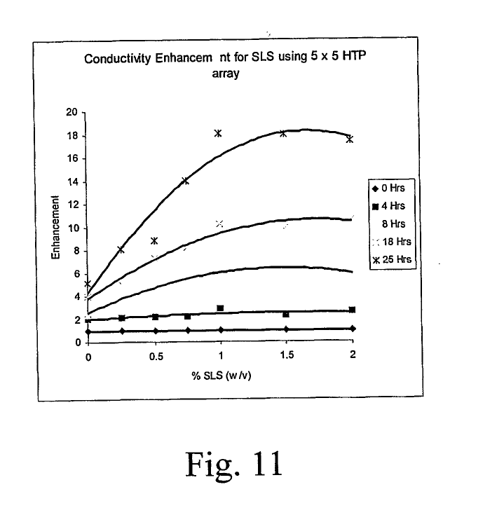

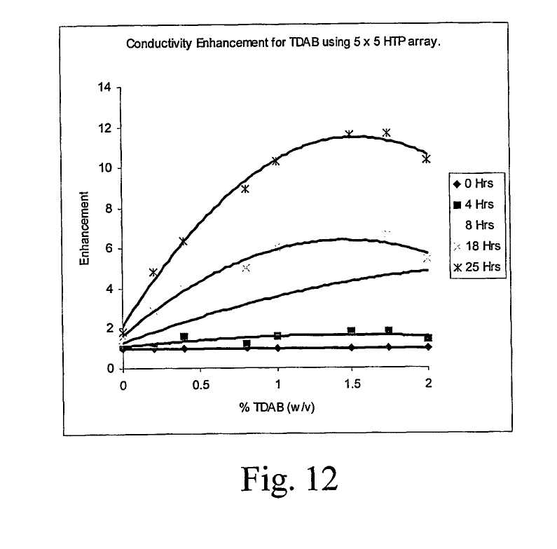

The conductivity enhancements at time t″ is then calculated as

$${E}_{t}^{C} = \frac{{I}_{t}}{{I}_{0}} ,$$

where Iₜ is the current across the skin at time ‘t’ and I₀ is the current across the skin at time 0. Conductivity enhancement for SLS (FIG. 11) and TDAB (FIG. 12) is plotted at various times for different concentrations between 0% (w/v) to 2% (w/v). The enhancement increases as a function of the SLS or TDAB concentration and reaches a maximum. The position of the maximum is a function of time.

b. Radiation Measurements.

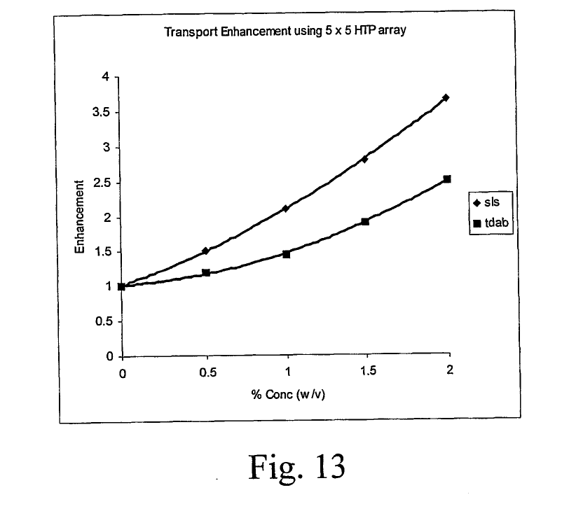

Radiolabelled mannitol was added to all formulations prepared in PBS at a concentration of 10 μCi/mL. The donor compartments were filled with these formulations each formulation was filled in 1 well. The skin was then incubated for 7 hrs. The solutions from the donor compartment were removed at the end of incubation period. The skin was then gently rinsed to free any mannitol that could be sticking to the surface of the skin. The skin was then cut and dissolved in 0.5 M Solvable, a tissue and gel solubilizer from Packard Chemicals at 60° C. overnight. A 500 μL sample was then talren and concentration of radiolabeled mannitol in this sample was then measured using a scintillation counter (Packard Tricarb 2000 CA). The transport enhancement at different test formulations is then calculated as

$${E}^{T} = \frac{{C}_{F}}{{C}_{C}} ,$$

where C_{F} is the radiation count for a particular test formulation and C_{C} is the radiation count for the control i.e. PBS alone without any enhancer. Transport enhancement for SLS and TDAB (FIG. 13) is plotted at various times for different concentrations between 0% (w/v) to 2% (w/v). The amount of mannitol transported increases monotonously as a function of the surfactant concentration.

4. Validation of HTP Method Against Results from Franz Diffusion Cells.

This example shows that the activity information for the surfactants extracted from the 10×10 HTP array or the 5×5 HTP array is qualitatively as well as quantitatively the same as that obtained from Franz diffusion cells. For this purpose transport and conductivity experiments were both performed with Franz diffusion cells for the same model enhancers in the manner described below.

a. Conductivity Experiments:

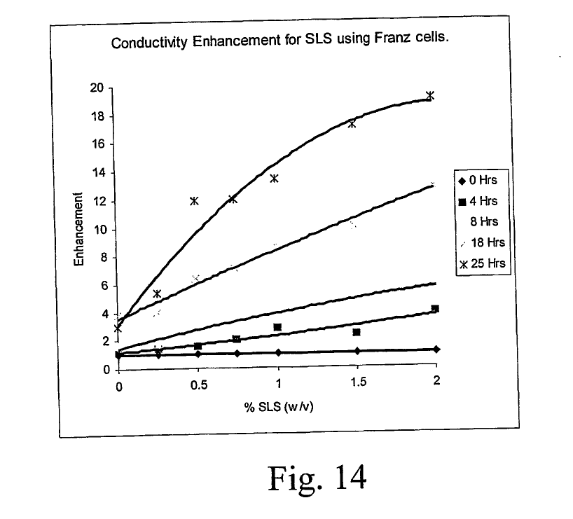

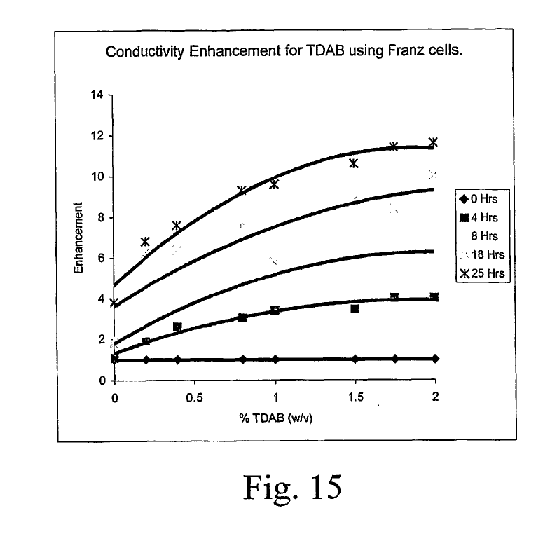

Pigskin samples, about 2-3 sq.cm, without any detectable scratches or abrasions were used for these experiments. Transdermal experiments were carried out using a vertical Franz diffusion cell (receiver volume=12 ml, area=2 cm²), which consists of a donor and a receiver compartment. A small stir bar and an Ag/AgCl disk electrode (E242 In-vivo Metrics) were added to the receiver chamber. In addition, the receiver chamber was filled with PBS. Pigskin was thawed and was mounted on the diffusion cell with the epidermis side facing up. The donor and the receiver compartments were clamped making sure there were no bubbles in the receiver chamber. Before each experiment, structural integrity of the skin was confirmed by measuring its conductivity. Skin samples with a resistivity less than 20 Kohm-cm² were assumed to be defective and not used. Skin conductivity was measured throughout the experiment to assess the effect of the formulation on skin structure. Effect of different formulations at different surfactant concentrations in the range 0 to 2% was tested on skin conductivity. Each formulation was prepared in PBS. Current across the skin was measured over a period of 24 hours. The enhancement of skin conductivity was calculated as

$${E}_{t}^{C} = \frac{{I}_{t}}{{I}_{0}} ,$$

where Iₜ is the current across skin at time t, I₀ being the current across skin at time 0. Conductivity enhancement for SLS (FIG. 14) and TDAB (FIG. 15) is plotted at various times for different concentrations between 0% (w/v) to 2% (w/v). The enhancement increases as a function of the SLS or TDAB concentration and reaches a maximum. The position of the maximum is a function of time.

b. Radiation Experiments.

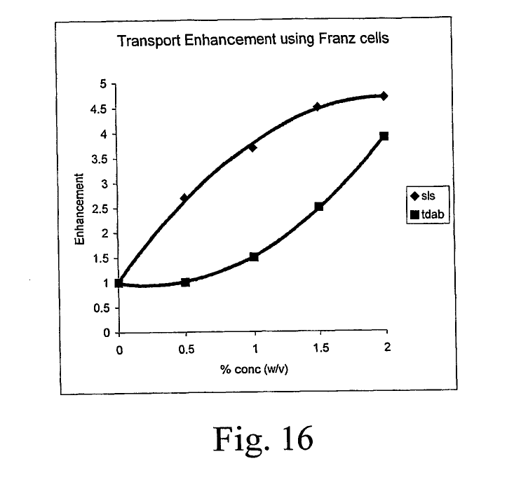

To assess the effect of formulations on skin permeability, radiolabeled mannitol (³H labeled) was added to the formulation at a concentration of 10 μCi/ml. The skin was incubated in the cells for 7 hrs. At the end of 7 hrs the skin was removed from the cell and dissolved in 0.5M Solvable, a gel and tissue solubilizer from Packard Chemicals, at 60° C. overnight. About 250 μL samples were then taken from the dissolved skin solution and concentration of radiolabeled mannitol was measured using a scintillation counter (Packard Tricarb 2000 CA). The enhancement of transdermal mannitol transport due to the formulations was calculated using the equation, E^{T}=C_{F}/C_{C}, where C_{F} is the radiation count for a particular formulation and C_{C} is the radiation count for the control which in this case is simply PBS with radiolabeled mannitol but no surfactant. Transport enhancement for SLS and TDAB (FIG. 16) is plotted at various times for different concentrations between 0% (w/v) to 2% (w/v). The amount of mannitol transported increases monotonously as a function of the surfactant concentration.

5. Data Analysis.

We now put together the data from Franz diffusion cells and the HTP arrays to see if it conveys the same information. The following observations can be made by looking at the data

a) The qualitative nature of enhancement as a function of surfactant concentration and exposure time is similar in case of Franz diffusion cells and HTP arrays (for both SLS and TDAB) for both conductivity and transport experiments.

b) The enhancement at each formulation at a given time (for both SLS and TDAB) is approximately the same within limits of experimental accuracy and skin variability in both, Franz diffusion cell and HTP arrays, in conductivity and transport experiments.

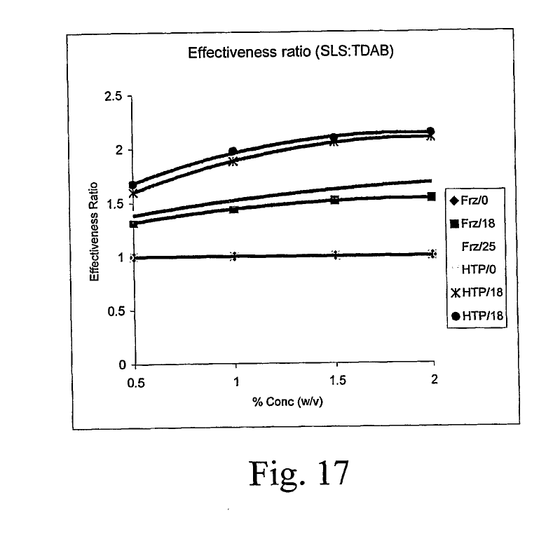

c) HTP screening can be used to determine the effectiveness of one enhancer over another. We plot the effectiveness of SLS over TDAB for Franz diffusion cell and for 10×10 HTP array (FIG. 17). It turns out that this effectiveness ratio (defined as the ratio of the enhancements of the two surfactants at a given concentration and given time) remains the same in both the geometries within limits of experimental accuracy and skin variability.

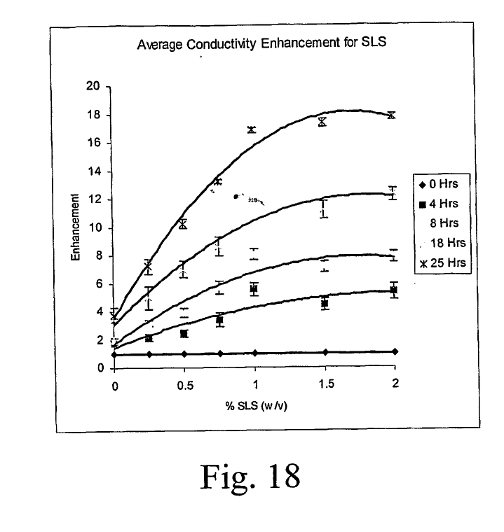

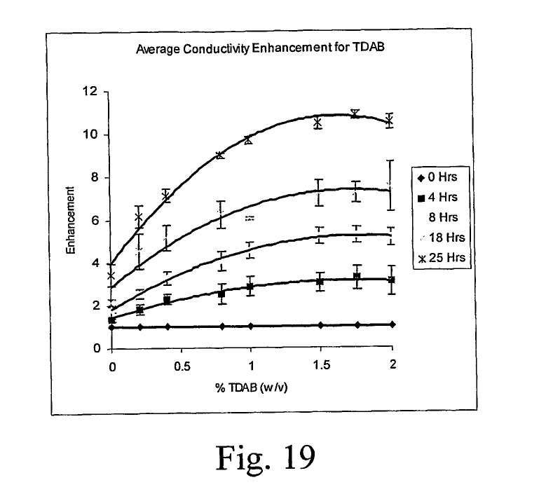

d) If we plot the average conductivity enhancement data at different concentrations for various times for all the three geometries it can be seen that this data is consistent within itself in limits of experimental accuracy and skin variability irrespective of the geometry of the cell for SLS (FIG. 18) as well as TDAB (FIG. 19). The error bars in FIGS. 18 and 19 correspond to the standard deviations in the conductivity enhancement obtained from Franz diffusion cells and the High Throughput arrays. The low standard deviation of all curves in FIGS. 18 and 19 show that the data obtained from the 5×5 array and 10×10 array is comparable to that obtained from Franz diffusion cells.

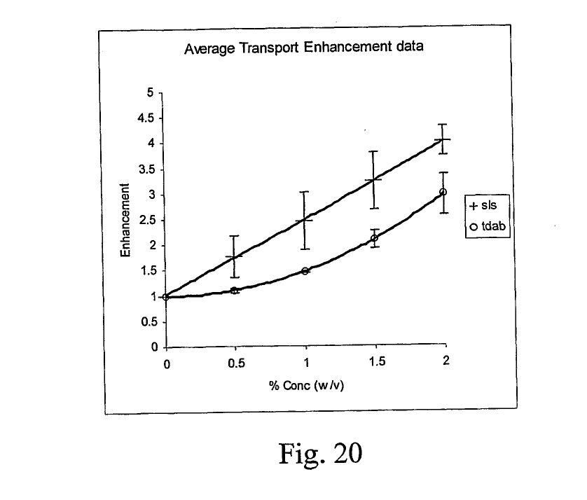

e) Similar conclusions can be made based on transport enhancement data shown in FIG. 20, where the error bars correspond to the standard deviations in the transport enhancement obtained from Franz diffusion cells and the High Throughput arrays. The low standard deviation of all curves in FIG. 20 show that the data obtained from the 5×5 array and 10×10 array is comparable to that obtained from Franz diffusion cells.

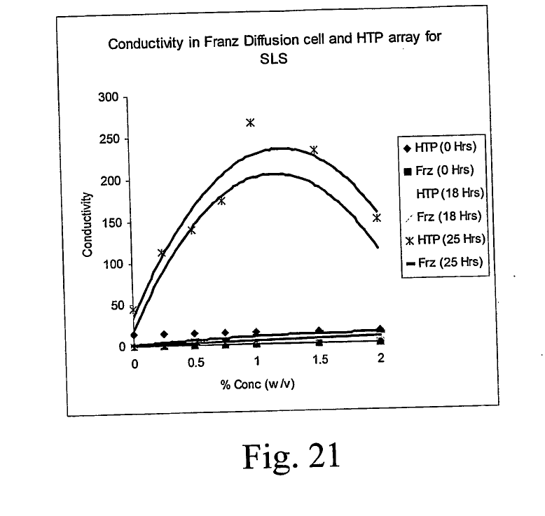

f) Conductivity is proportional to the ratio of current to the area in any two assemblies at a constant skin thickness and applied voltage. If we plot this ratio of current over area it is seen that the resolution obtained with the HTP arrays is significantly better than in Franz diffusion cells (FIG. 21). Moreover, the resolution increases as we go down to finer hole sizes. This is of significant importance in these experiments where we deal with high variabilities in the inherent skin permeability itself.

The following references are incorporated herein by reference:

Bronaugh, R. L., Determination of Percutaneous Absorption by In Vitro Techniques, In Percutaneous Absorption; Mechanisms-Methodology-Drug Delivery, R. L. Bronaugh, Maibach, H. I., Editor, Marcel Deldcer Inc.: New York, Basel, 1989,.

Crank, J., Mathematics of Diffusion, Oxford Publishers, 1975.

Elias, P. M., Cooper, E. R., Korc, A. and Brown, B. E. Percutaneous Transport in Relation to Stratum Corneum Structure and Lipid Composition. J. Invest. Dermatol. 76: 297-301 (1981).

Johnson, M. E, Biophysical Aspects of Transdermal Drug Delivery and Chemical Enhancement, in Chemical Engineering. 1996, Massachusetts Institute of Technology: Cambridge. p. 260.

Johnson, M. E., Berk, D. A., Blankschtein, D. and Langer, R. Lateral Dlffusion of Small Compounds in Human Stratum Corneum and Model Lipid Bilayer Systems. Biophysical J 71: 26562668 (1996).

Mitragotri, S. In Situ Determination of Partition and Diffusion Coefficients in the Lipid Bilayers of the Stratum Corneum. Pharm. Res. Submitted: (2000).

Mitragotri, S., Johnson, M. E., D., B. and Langer, R. A Theoretical Analysis of Partitioning, Difflusion, and Permeation Across lipid bilayers. Biophys. J 77: 1268-1283 (1999).

Ng, S., Goodson, B., Elrardt, R., Moos, W. H., Siani, M. and Winter, J. Combinatorial Discovery Process Yields Antimicrobial Peptides. Prog. Med. Chem. 7: 1781-5 (1999).

Verdine, G. L. The Combinatorial Chemistry of Nature. Nature. 384: 11-13 (1996).

Zhang, B., Salituro, G., Szalkowsld, D., Li, Z., Zhang, Y., Royo, I., Vielella, D., Diez, M. T., Pelaez, F., Ruby, C., Kendall, R. L., MAo, X., Griffin, P., Calaycacy, J., Zierath J R, H., Smith, R. G. and Moller, D. E. Discovery of a Small Molecule Insulin Mimetic with Antidiabetic Activity in Mice. Science. 284: 974-7 (1999).

What is claimed is:

1. A method for performing high throughput assays of drug delivery formulations, the method comprising:
i) securing a test membrane to a device comprising a donor plate, the donor plate including a plurality of donor wells formed by donor holes passing through the donor plate;
ii) introducing a formulation into each donor well, the formulation including a test substance and an inert medium;
iii) evaluating a characteristic of the test substance that remains in the donor well or migrates into the test membrane;

2. The method of claim 1 wherein the device further comprises a receiver plate including a plurality of receptor wells corresponding to the donor wells of the donor plate and the test membrane is secured between the donor plate and the receiver plate.

3. The method of claim 2 further comprising the step of evaluating a characteristic of the test substance that migrates through the membrane into the receptor wells.

4. The method of claim 1, said donor holes having a diameter of about 40 microns to about 10 mm.

5. The method of claim 1 wherein the volume of the donor wells is about 1 to 500 μl.

6. The method of claim 1 wherein the test substance is a drug or its analog.

7. The method of claim 1 wherein the drug or its analog is a radioactive or fluorescent substance.

8. The method of claim 1 wherein the formulation includes one or more permeability enhancers.

9. The method of claim 1 wherein the inert medium is an ointment, cream, gel, solution or lotion.

10. The method of claim 1 wherein the characteristic of the test substance that remains in the donor well is its concentration.

11. The method of claim 1 wherein the characteristic of the test substance that migrates into the test membrane is its radioactivity, fluorescence or enhancement of membrane conductivity.

12. The method of claim 1 wherein the evaluating step recurs at one or more periodic intervals of less than about 24 hours.

13. The method of claim 1 wherein the evaluating step occurs no later than 8 hours after introducing the formulation to the donor well.

14. The method of claim 1 wherein the device further comprises two electrodes to measure current across the membrane.

15. The method of claim 14 wherein the characteristic of the test substance that migrates into the test membrane is the enhancement of membrane conductivity.

16. The method of claim 1 wherein the test membrane is mammalian skin or mucosa.

17. A device for conducting high throughput assays of drug formulations, the device comprising:
i) a donor plate, the donor plate including a plurality of donor wells formed by donor holes passing through the donor plate;
ii) means for securing a test membrane to the donor plate, whereby one or more donor wells are sealed at one end of the well and transfer of one or more substances to the membrane can occur; and
iii) one or more electrodes to measure current across a portion of the test membrane, said portion sealing an individual donor well.
